(19) United States
(12) Patent Application Publication
(10) Pub. No.: US 2021/0015753 A1
(43) Pub. Date: Jan. 21, 2021

(54) FREEZE-DRIED MULTIPARTICULATE SOLID DOSAGE FORM

(71) Applicant: DSM IP ASSETS B.V., Heerlen (NL)

(72) Inventors: Maria EBENRETH, Kaiseraugst (CH); Denis KARPUKHIN, Kaiseraugst (CH); Marcel MOONEN, Kaiseraugst (CH)

(21) Appl. No.: 16/766,893

(22) PCT Filed: Nov. 22, 2018
(86) PCT No.: PCT/EP2018/082156

§ 371 Date: May 26, 2020

(30) Foreign Application Priority Data
Nov. 27, 2017 (EP) 17203799.6

(51) Int. Cl. A61K 9/19; A61K 9/50; A61K 31/202; A23L 33/12

(52) U.S. Cl. CPC A61K 9/19; A61K 9/5042; A23L 33/12; A61K 31/202; A61K 9/5089

(57) ABSTRACT

The present invention relates to a freeze dried multiparticulate solid dosage form (1) that contains a plurality of microcapsules (2) having a core (2a) and a shell (2b) that are embedded in a freeze-dried, edible matrix (3). Microcapsules (2) contain an active ingredient which may be a pharmaceutical drug and/or a micronutrient. The solid dosage form may be a tablet, a wafer or even a crispy snack. A method for preparing such dosage forms is also disclosed.

### TECHNICAL FIELD

The present invention relates to the manufacture of multiparticulate solid dosage forms for oral administration.

Multiparticulate solid dosage forms are produced by compressing pellets together with customary excipients and additives to produce tablets. Said pellets contain an active ingredient. Pellets may also have a functional coating to control the release of the active ingredient.

### BACKGROUND OF THE INVENTION

WO 2013/092589 discloses a multiple unit pellet tablet formulation for oral administration. On page 4 of WO 2013/092589, issues relating to the compression of pellets are discussed.

Compressing pellets entails the risk that the functional coating of the pellets will be damaged, resulting in considerable risks for the patient, as described in US 2010/247647, paragraph [0006].

A typical tablet press applies a pressure of approx. 75 kN/cm². So far, there is no solution to prevent damage or to avoid fracturing of the pellets when such high pressures are applied.

Microcapsules are smaller than pellets. However, similar problems occur when compressing microcapsules together with customary excipients and additives to give a multiparticulate solid dosage form for oral consumption.

WO 2009/010305 relates to the reduction of extrusion loss when pressing a formulation of a lipophilic health ingredient to tablets, said lipophilic health ingredient being encapsulated by modified starch.

Thus, there is a need for an improved method for producing multiparticulate solid dosage forms.

### SUMMARY OF THE INVENTION

Microcapsules are defined as small particles of solids, or droplets of liquids, inside a thin coating of a shell material such as beeswax, starch, gelatin or polyacrylic acid. They are used, for example, to protect against oxidation and/or to control the rate of release of an active ingredient such as an enzyme, a flavor, a nutrient, a drug, etc.

The problem to be solved by the present invention is avoiding cracks, holes, etc. in the shell of the microcapsules. Such damage typically occurs when a mixture comprising microcapsules and customary excipients is compressed to produce a multiparticulate solid dosage form.

Cracks in the shell of microcapsules may result in leakage of the active ingredient which is encapsulated by said shell. Thus, another problem to be solved by the present invention is avoiding leakage of the active ingredient when producing a multiparticulate solid dosage form.

Some active ingredients have a bad taste or off-flavor. The leakage of such an active ingredient renders the solid dosage form unusable. Thus, another problem to be solved by the present invention is to lower the number of defective products when manufacturing multiparticulate solid dosage forms.

Furthermore, patient compliance with a solid dosage form having an off-flavor is low. Thus, another problem to be solved by the present invention is increasing patient compliance with multiparticulate solid dosage forms which comprise an active ingredient that has an off-flavor or that becomes smelly upon oxidation.

Surprisingly, multiparticulate solid dosage forms can be manufactured by freeze-drying a mixture comprising water, an edible matrix and microcapsules.

When compression by a tablet press is replaced by freeze-drying, the above-mentioned problems do not occur.

### DETAILED DESCRIPTION OF THE INVENTION

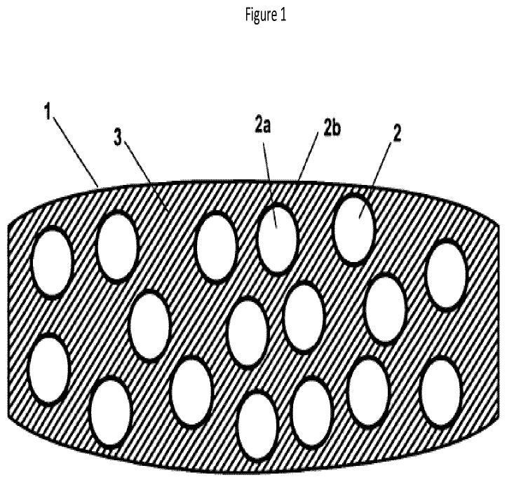

FIG. 1 schematically illustrates a multiparticulate solid dosage form (1) that contains a plurality of microcapsules (2) having a core (2a) and a shell (2b) that are embedded in a freeze-dried, edible matrix (3).

In the context of the present invention, cells (such as bacterial cells) and viruses are not encompassed by the term “microcapsules”.

In the context of the present invention, the term “solid dosage form” is limited to a dosage form that is administered orally or that can be eaten. This includes small dosage forms that can be swallowed as a whole, similar to a tablet. However, it also includes dosage forms which are too big to be swallowed without chewing. The person skilled in the art understands that the size of the dosage form needs to be adapted to the size and nature of the microcapsules that are embedded in the freeze-dried, edible matrix. If microcapsules with a functional coating are used, the dosage form should not be chewed as chewing could damage the functional coating. In such cases, the dosage form must be small enough to be swallowed without chewing.

In a preferred embodiment of the invention, the “multiparticulate solid dosage form” of the invention has a volume of at least 0.36 ml (corresponding to the volume capacity of an empty two-piece hard capsules size 2), preferably of at least 0.68 ml (corresponding to size 0), more preferably of at least 0.9 ml (corresponding to size 00), even more preferably of at least 1 ml (corresponding to size 00E) and most preferably of at least 1.37 ml (corresponding to size 000).

In the context of the present invention, the term “functional coating” refers to a layer that covers shell (2b) of microcapsule (2), such the active ingredient is released in a controlled manner. The microcapsules according to the invention have preferably no functional coating.

The multiparticulate solid dosage form according to the invention comprises a freeze-dried, edible matrix. Like any other freeze-dried product, the freeze-dried, edible matrix has a relative low density. In one embodiment of the invention, the multiparticulate solid dosage form according to the invention has a density of less than 1 g/cm³, measured at room temperature. In a preferred embodiment of the invention, the multiparticulate solid dosage form according to the invention has a density of less than 0.8 g/cm³, measured at room temperature.

In some embodiments, the freeze-dried, edible matrix is crispy. Patient compliance may thus be enhanced by providing a relative large dosage form resembling a crispy form, such as in a cracker or a wafer. Thus, in one embodiment of the invention, the multiparticulate solid dosage form has a surface of at least 10 cm², preferably of at least 15 cm² and most preferably of at least 20 cm², measured by BET (preferably according to ISO 9277:1995).

In some embodiments of the invention, the weight ratio between the freeze-dried, edible matrix and said microcapsules is at least 10:1, preferably at least 20:1 and most preferably at least 30:1.

In a preferred embodiment of the invention, the weight ratio between the freeze-dried, edible matrix and said microcapsules is from 5:1 to 50:1, preferably from 5:1 to 40:1 and most preferably from 5:1 to 30:1.

Microcapsules according to the invention have typically an average diameter of from 1 μm to about 2000 μm. Due to this small size, hundreds of microcapsules may be embedded in the freeze-dried, edible matrix. The freeze-dried, edible matrix according to the invention comprises preferably at least 200 microcapsules, more preferably at least 250 microcapsules, and most preferably more than 300 microcapsules.

Thus, a preferred embodiment of the present invention relates to a multiparticulate solid dosage form comprising at least 200 microcapsules, wherein said microcapsules are embedded in a freeze-dried, edible matrix, characterized in that said microcapsules do not have a functional coating, and characterized in that said multiparticulate solid dosage form
i. has a total surface of at least 10 cm², measured by BET (ISO 9277:1995), and/or
ii. has a density of less than 0.8 g/cm³.

The core (2a) of microcapsule (2) comprises at least one active ingredient. Depending on the nature of the active ingredient, the core may be liquid, solid or mixtures thereof. In one embodiment of the invention, the microcapsules encapsulate at least one hydrophobic or hydrophilic compound. Said compound may be a pharmaceutical drug and/or a micronutrient.

Examples of pharmaceutical drugs are opioids including μ-opioid receptor agonists such as alfentanil, buprenorphine, codeine, fentanyl, hydrocodone, hydromorphone, levomethadone, methadone, morphine, nalbuphine oxycodone, oxymorphone, pethidine, piritramid, remifentanil, sufentanil, tapentadol, tilidin, tramadol, and pharmaceutically acceptable salts thereof.

Thus, one embodiment of the present invention relates to a multiparticulate solid dosage form comprising microcapsules, wherein said microcapsules are embedded in a freeze-dried, edible matrix, characterized in that said microcapsules have preferably a functional coating, and

characterized in that said multiparticulate solid dosage form
i. has a total surface of at least 10 cm², measured by BET (ISO 9277:1995), and/or
ii. has a density of less than 0.8 g/cm³
   and characterized in that said microcapsules encapsulate a pharmaceutical drug such as an opioid, said opioid preferably selected from the group consisting of alfentanil, buprenorphine, codeine, fentanyl, hydrocodone, hydromorphone, levomethadone, methadone, morphine, nalbuphine oxycodone, oxymorphone, pethidine, piritramid, remifentanil, sufentanil, tapentadol, tilidin, tramadol, and pharmaceutically acceptable salts thereof.

Examples of micronutrients are vitamins, minerals, plant extracts, or oils such as microbial or marine oils.

Oil produced by a microorganism or obtained from a microbial cell is referred to as a “microbial oil”. Oil produced by algae and/or fungi is referred to as an algal and/or a fungal oil, respectively.

As used herein, a “microorganism” refers to organisms such as algae, bacteria, fungi, protist, yeast, and combinations thereof, e.g., unicellular organisms. A microorganism includes but is not limited to, golden algae (e.g., microorganisms of the kingdom Stramenopiles); green algae; diatoms; dinoflagellates (e.g., microorganisms of the order Dinophyceae including members of the genus Crypthecodinium such as, for example, Crypthecodinium cohnii or C. cohnii); microalgae of the order Thraustochytriales; yeast (Ascomycetes or Basidiomycetes); and fungi of the genera Mucor, Mortierella, including but not limited to Mortierella alpina and Mortierella sect. schmuckeri, and Pythium, including but not limited to Pythium insidiosum.

In one embodiment, the microorganisms are from the genus Mortierella, genus Crypthecodinium, genus Thraustochytrium, and mixtures thereof. In a further embodiment, the microorganisms are from Crypthecodinium Cohnii. In a further embodiment, the microorganisms are from Mortierella alpina. In a still further embodiment, the microorganisms are from Schizochytrium sp. In yet an even further embodiment, the microorganisms are selected from Crypthecodinium Cohnii, Mortierella alpina, Schizochytrium sp., and mixtures thereof.

In a still further embodiment, the microorganisms include, but are not limited to, microorganisms belonging to the genus Mortierella, genus Conidiobolus, genus Pythium, genus Phytophthora, genus Penicillium, genus Cladosporium, genus Mucor, genus Fusarium, genus Aspergillus, genus Rhodotorula, genus Entomophthora, genus Echinosporangium, and genus Saprolegnia.

In an even further embodiment, the microorganisms are from microalgae of the order Thraustochytriales, which includes, but is not limited to, the genera Thraustochytrium (species include arudimentale, aureum, benthicola, globosum, kinnei, motivum, multirudimentale, pachydermum, proliferum, roseum, striatum); the genera Schizochytrium (species include aggregatum, limnaceum, mangrovei, minutum, octosporum); the genera Ulkenia (species include amoeboidea, kerguelensis, minuta, profunda, radiate, sailens, sarkariana, schizochytrops, visurgensis, yorkensis); the genera Aurantiacochytrium; the genera Oblongichytrium; the genera Sicyoidochytium; the genera Parientichytrium; the genera Botryochytrium; and combinations thereof. Species described within Ulkenia will be considered to be members of the genus Schizochytrium. In another embodiment, the microorganisms are from the order Thraustochytriales. In yet another embodiment, the microorganisms are from Thraustochytrium. In still a further embodiment, the microorganisms are from Schizochytrium sp.

In certain embodiments, the oil can comprise a marine oil. Examples of suitable marine oils include, but are not limited to, Atlantic fish oil, Pacific fish oil, or Mediterranean fish oil, or any mixture or combination thereof. In more specific examples, a suitable fish oil can be, but is not limited to, pollack oil, bonito oil, pilchard oil, tilapia oil, tuna oil, sea bass oil, halibut oil, spearfish oil, barracuda oil, cod oil, menhaden oil, sardine oil, anchovy oil, capelin oil, herring oil, mackerel oil, salmonid oil, tuna oil, and shark oil, including any mixture or combination thereof. Other marine oils suitable for use herein include, but are not limited to, squid oil, cuttle fish oil, octopus oil, krill oil, seal oil, whale oil, and the like, including any mixture or combination thereof.

Manufacturing a multiparticulate solid dosage form that comprises omega-3 fatty acids such as docosahexaenoic acid (DHA) is particularly challenging because of the fishy off-flavor which occurs upon oxidation.

Thus, a preferred embodiment of the present invention relates to a multiparticulate solid dosage form comprising at least 200 microcapsules, wherein said microcapsules are embedded in a freeze-dried, edible matrix, characterized in that said microcapsules encapsulate at least one micronutrient such as a vitamin, mineral, plant extract, oil or mixtures thereof.

Freeze-drying hydrophobic liquids is a challenge. If one is trying to freeze-dry a substance with oil, certain parts of the oil will sublimate first, leaving behind a slicker consistency. Surprisingly, this difficulty can be avoided when using microcapsules.

Particularly good results are achieved when shell (2b) comprises or consists of a hydrocolloid such as modified starch, gelatin, polyphosphate, gum arabic, alginate, chitosan, carrageenan, pectin, carboxymethylcellulose or mixtures thereof.

Thus, a preferred embodiment of the present invention relates to a multiparticulate solid dosage form comprising at least 200 microcapsules, wherein said microcapsules are embedded in a freeze-dried, edible matrix, characterized in that said microcapsules
i. do not have a functional coating, and
ii. encapsulate hydrophobic liquid comprising a polyunsaturated fatty acid, and
iii. have at least one shell, said shell comprising or consisting of at least one hydrocolloid such as a modified starch, gelatin, polyphosphate, gum arabic, alginate, chitosan, carrageenan, pectin, carboxymethylcellulose or mixtures thereof.

In a preferred embodiment, the polyunsaturated fatty acid is in the form of a free fatty acid, salt, fatty acid ester (e.g., methyl or ethyl ester), monoacylglycerol (MAG), diacyglycerol (DAG) triacylglycerol (TAG), and/or phospholipid (PL) or mixtures thereof.

In a preferred embodiment, the polyunsaturated fatty acid is an omega-3 fatty acid, an omega-6 fatty acid, or mixtures thereof. In the most preferred embodiment, the the polyunsaturated fatty acid is docosahexaenoic acid (DHA).

In a particularly preferred embodiment of the invention, the multiparticulate solid dosage form comprises microcapsules as disclosed in WO 03/086104. The content of WO 03/086104 is hereby incorporated by reference. In said embodiment, the multiparticulate solid dosage form comprises agglomerations of primary microcapsules, each individual primary microcapsule having a primary shell and the agglomeration being encapsulated by an outer shell. Preferably, the primary shell and/or the outer shell comprise gelatin, polyphosphate, gum arabic, alginate, chitosan, carrageenan, pectin, carboxymethylcellulose or mixtures thereof. Said agglomerations are particularly suitable for freeze-drying a mixture comprising water, an edible matrix and microcapsules. It is known that such agglomerations can be obtained by coacervation. Thus, said primary microcapsules are preferably coacervates.

Thus, in a preferred embodiment of the invention, the multiparticulate solid dosage form comprises microcapsules, wherein said microcapsules are embedded in a freeze-dried, edible matrix, and
wherein said multiparticulate solid dosage form comprises agglomerations of primary microcapsules, each individual primary microcapsule having a primary shell and the agglomeration being encapsulated by an outer shell, and
wherein the primary shell and/or the outer shell comprise gelatin, polyphosphate, gum arabic, alginate, chitosan, carrageenan, pectin, carboxymethylcellulose or mixtures thereof, and
wherein said primary shell encapsulates a hydrophobic liquid comprising preferably a polyunsaturated fatty acid.

In said embodiment, the polyunsaturated fatty acid is preferably in the form of a free fatty acid, salt, fatty acid ester (e.g., methyl or ethyl ester), monoacylglycerol (MAG), diacyglycerol (DAG) triacylglycerol (TAG), and/or phospholipid (PL) or mixtures thereof, wherein said polyunsaturated fatty acid is preferably an omega-3 fatty acid, an omega-6 fatty acid, or mixtures thereof.

Another embodiment of the invention relates to a multiparticulate solid dosage form comprising at least 200 microcapsules, wherein said microcapsules are embedded in a freeze-dried, edible matrix, characterized in that said microcapsules are microcapsules according to any of claims 1-24 of WO 03/086104.

To enhance patient compliance, the edible matrix of the invention is preferably fruit-flavored and/or contains sugar. In a particularly preferred embodiment of the invention, a fruit puree such as banana puree, apple puree or a mixture thereof is mixed with microcapsules before freeze-drying the obtained mixture. Alternatively, a composition comprising yoghurt is mixed microcapsules before freeze-drying the obtained mixture.

Thus, a preferred embodiment of the present invention relates to a multiparticulate solid dosage form (1) comprising microcapsules (2), wherein said microcapsules (2) are embedded in a freeze-dried, edible matrix (3),
characterized in that said freeze-dried, edible matrix (2) comprises fruit puree and/or sugar, and
characterized in that said microcapsules (2) preferably do not have a functional coating, and
characterized in that said microcapsules have a core (2a) and shell (2b), and
characterized in that said shell (2b) comprises at least one hydrocolloid such as modified starch, gelatin, polyphosphate, gum arabic, alginate, chitosan, carrageenan, pectin, carboxymethylcellulose or mixtures thereof, and
characterized in that said shell (2b) encapsulates a vitamin, a mineral, a plant extract, an oil or mixtures thereof and preferably encapsulates a hydrophobic liquid comprising preferably a polyunsaturated fatty acid, and
characterized in that said freeze-dried, edible matrix (2) embeds preferably at least 200 microcapsules (2).

An even more preferred embodiment of the present invention relates to a multiparticulate solid dosage form (1) comprising microcapsules (2), wherein said microcapsules (2) are embedded in a freeze-dried, edible matrix (3),
characterized in that said freeze-dried, edible matrix (2) comprises fruit puree and/or sugar, and
characterized in that said microcapsules (2) do not have a functional coating, and
characterized in that said microcapsules have a core (2a) and shell (2b), and
characterized in that said shell (2b) comprises at least one hydrocolloid such as modified starch, gelatin, polyphosphate, gum arabic, alginate, chitosan, carrageenan, pectin, carboxymethylcellulose or mixtures thereof, and
characterized in that said shell (2b) encapsulates a vitamin, a mineral, a plant extract, an oil or mixtures thereof and preferably encapsulates a hydrophobic liquid comprising an omega-3 fatty acid, an omega-6 fatty acid and/or mixtures thereof, and
characterized in that said freeze-dried, edible matrix (2) embeds preferably at least 200 microcapsules (2).

In said even more preferred embodiment, the omega-3 fatty acid and/or the omega-6 fatty acid may be in the form of a free fatty acid, a salt, a fatty acid ester (e.g., methyl or ethyl ester), a monoacylglycerol (MAG), a diacyglycerol (DAG), a triacylglycerol (TAG), a phospholipid (PL) or mixtures thereof.

In one embodiment, the present invention relates to the use of microcapsules being embedded in a freeze-dried, edible matrix for enhancing patient compliance with active ingredients which have preferably an off-flavor such as a fishy off-flavor. In another embodiment, the present invention relates to a multiparticulate solid dosage form as described herein for use in the enhancement of patient compliance when treating said patient with an oral dosage form, wherein said oral dosage form comprises at least one active ingredient which has preferably an off-flavor such as a fishy off-flavor.

Multiparticulate solid dosage forms according to the invention are produced by freeze-drying a mixture comprising water, an edible matrix and microcapsules. The person skilled in the art is familiar with freeze-drying technique: material is frozen before reducing the surrounding pressure to allow the frozen water in the material to sublime directly from the solid phase to the gas phase.

Typically, a freeze-drying apparatus has walls and one or more shelves carrying the product to be freeze-dried. In a preferred embodiment, the product to be freeze-dried comprises microcapsules as described in the previous sections, and most preferably also fruit puree such as banana puree, apple puree or a mixture thereof. Therefore, the present invention also relates to a freeze-drying apparatus having walls and at least one shelf, tray or belt carrying the product to be freeze-dried, wherein the product to be freeze-dried comprises microcapsules, characterized in that said microcapsules encapsulate at least one pharmaceutical drug and/or at least one micronutrient.

Thus, the method for producing multiparticulate solid dosage forms comprises the step of putting a mixture comprising water, an edible matrix and microcapsules on a shelf, on a tray or on a belt, wherein said shelf, tray or belt is inserted into a freeze-drying apparatus. In a preferred embodiment, the microcapsules and/or the edible matrix as described herein is put on said shelf, tray or belt.

Any suitable packaging material can be used to package the multiparticulate solid dosage forms according to the invention. In a preferred embodiment of the invention, the multiparticulate solid dosage forms are packaged in boxes, bottles, blisters or bags. Not preferred are vials such as vials used for packaging of lyophilized powder. In the context of the present invention, a particle of a lyophilized powder is not a multiparticulate solid dosage form.

One embodiment of the present invention relates to a package comprising at least one multiparticulate solid dosage form as herein described, wherein said package is not a vial.

In a preferred embodiment of the invention, several (i.e. more than one) multiparticulate solid dosage forms as described herein are packed into a reclosable bottle or into a resealable bag. Thus, the present invention also relates to a multidose product comprising at least 10, preferably at least 20, most preferably at least 30 multiparticulate solid dosage forms as described herein, wherein said multidose product is reclosable or resealable. Preferably, said multidose product is a reclosable bottle or a resealable bag. Surprisingly, such multidose product has an excellent in use stability even if the therein contained oral dosage forms comprise an active ingredient which has an off-flavor such as a fishy off flavor.

### EXAMPLES

The present invention is further illustrated by the following examples.

### Example 1

In example 1, docosahexaenoic acid (DHA) has been chosen as model substance. DHA is an omega-3 fatty acid. Like most other omega-3 fatty acids, DHA is prone to oxidation. Upon oxidation, DHA gets a fishy off-flavor that is easily recognized upon consumption, even at very low quantities.

In example 1, MEG-3® DHA K Powder (available from DSM®) is mixed with banana puree. Droplets having different sizes are formed from the thus obtained mixture. The droplets are then freeze-dried to obtain crispy droplets resembling brownish cookies. The results of sensory testing directly after freeze-drying (TO) and after three months storage at room temperature (T3) are given in Table 1.

No fishy taste has been recognized by the sensory panel even as the amount of DHA is increased from 40 mg to 250 mg (cf. trials 2 and 3) or even as the amount of matrix material is decreased by a factor 10 (cf. trials 2 and 4).

MEG-3® DHA K Powder is made of microcapsules encapsulating DHA. Thus, example 1 illustrates that the shell of said microcapsules is not damaged when banana puree comprising MEG-3® DHA K Powder is freeze-dried.

As a comparison, MEG-3® DHA K Powder is vigorously grinded and crashed with pestle and mortar. A fishy off-flavor due to the damages in the shell of the microcapsules is instantaneously noted.

TABLE 1

| | | | desired | | | | approx. | | | caloric | |
|---|---|---|---|---|---|---|---|---|---|---|---|
| HBS | | | addition | | | | weight ratio | | | value per | |
| Trial | | active | level/serving | | Serving | | matrix: | Sensory | | portion | storage |
| No | Formulation | ingredient | amount | Unit | size g | Matrix | microcapsules | T0 | T3 | (10 g) | condition |
| 1 | control | DHA | 0.0 | mg | 10 | Banana | n.a. | typical banana | typical banana | 57 | Room |
| | | | | | | puree | | taste/aroma | taste/aroma | | temperature |
| 2 | FB 1 | DHA | 40.0 | mg | 10 | Banana | 50:1 | typical banana | typical banana | 57 | Room |
| | | | | | | puree | | taste/aroma, | taste/aroma, | | temperature |
| | | | | | | | | no fishy taste | no fishy taste | | |
| 3 | FB 11 | DHA | 250.0 | mg | 10 | Banana | 7:1 | typical banana | typical banana | 69 | Room |
| | | | | | | puree | | taste/aroma, | taste/aroma, | | temperature |
| | | | | | | | | no fishy taste | no fishy taste | | |
| 4 | DS 1 | DHA | 40.0 | mg | 1 | Banana | 5:1 | fade away of | fade away of | 80 | Room |
| | | | | | | puree | | banana | banana | | temperature |
| | | | | | | | | taste/aroma, | taste/aroma, | | |
| | | | | | | | | no fishy taste | no fishy taste | | |

### Example 2

In example 2, fruit-flavored matrices are tested aiming to enhance patient compliance.

To avoid the need of a formal phase I study, food approved model substances are being used as model substances. All model substances are available at DSM®.

Crispy or chewy droplets are obtained when a suitable amount of a matrix containing the active ingredients is freeze-dried.

The results of example 2 are given in Table 2.

TABLE 2

| HBS | active ingredient | | | |
|---|---|---|---|---|
| Trial | active | | | Sensory |
| No | ingredient | source | Matrix | T0 |
| 5 | trans-resveratrol | resVida ® | apple puree, | typical fruit |
| | | | juice | taste/aroma |
| | | | concentrate | |
| 6 | compounds to | fruitflow ® | apple puree, | typical fruit |
| | maintain healthy | | coconut milk | taste with |
| | platelet | | | coconut |
| | aggregation | | | flavour |
| | and improve | | | |
| | blood flow | | | |
| 7 | Lutein Zeaxanthin | FloraGLO ® | mango puree | typical |
| | | OPTISHARP ® | maltodextrin | mango |
| | | | | taste/aroma |
| 8 | vitamins, | Quali-Blends ® | banana puree | typical |
| | carotenoids, | | apple puree | banana |
| | minerals | | | taste/aroma |

1. Method of producing a multiparticulate solid dosage form, wherein a mixture comprising water, an edible matrix and microcapsules is freeze-dried.

2. Method according to claim 1, wherein said mixture comprises agglomerations of primary microcapsules, each individual primary microcapsule having a primary shell and the agglomerations being encapsulated by an outer shell, and wherein said primary microcapsules are preferably coacervates.

3. Multiparticulate solid dosage form comprising microcapsules, wherein said microcapsules are embedded in a freeze-dried, edible matrix.

4. Multiparticulate solid dosage form according to claim 3, wherein said multiparticulate solid dosage form comprises at least 200 microcapsules and/or wherein said microcapsules have no functional coating.

5. Multiparticulate solid dosage form according to claim 3, wherein the weight ratio between said freeze-dried, edible matrix and said microcapsules is from 5:1 to 50:1, preferably from 5:1 to 40:1 and most preferably from 5:1 to 30:1.

6. Multiparticulate solid dosage form according to claim 3, wherein said multiparticulate solid dosage form comprises agglomerations of primary microcapsules, each individual primary microcapsule having a primary shell and the agglomerations being encapsulated by an outer shell, and wherein said primary microcapsules are preferably coacervates.

7. Method according to claim 2, wherein the primary shell and/or the outer shell comprise gelatin, polyphosphate, gum arabic, alginate, chitosan, carrageenan, pectin, carboxymethylcellulose or mixtures thereof.

8. Method according to claim 1 wherein said multiparticulate solid dosage form has a volume of at least 0.36 ml, preferably of at least 0.68 ml, more preferably of at least 0.9 ml, even more preferably of at least 1 ml and most preferably of at least 1.37 ml.

9. Method according to claim 1, wherein said microcapsules encapsulate a pharmaceutical drug and/or a micronutrient.

10. Method according to claim 1, wherein said microcapsules encapsulate a hydrophobic liquid comprising preferably a polyunsaturated fatty acid.

11. Method or a multiparticulate solid dosage form according to claim 9, wherein the micronutrient is a polyunsaturated fatty acid and wherein the polyunsaturated fatty acid is preferably in the form of a free fatty acid, a salt, an ester, a monoacylglycerol (MAG), a diacylglycerol (DAG), a triacylglycerol (TAG), a phospholipid (PL) or mixtures thereof.

12. Method or a multiparticulate solid dosage form according to claim 10, wherein the polyunsaturated fatty acid is an omega-3 fatty acid, an ester of an omega-3 fatty acid, an omega-6 fatty acid, an ester of an omega-6 fatty acid or mixtures thereof.

13. Multiparticulate solid dosage form according to claim 3 for use in the enhancement of patient compliance when treating said patient with an oral dosage form, wherein said oral dosage form comprises at least one active ingredient which has preferably an off-flavor such as a fishy off-flavor.

14. Use, method or multiparticulate dosage form according to claim 1, wherein said freeze-dried, edible matrix comprises sugar and/or wherein said freeze-dried, edible matrix is fruit-flavored.

15. Multidose product comprising at least 10 multiparticulate solid dosage forms according to claim 3, wherein said multidose product is reclosable or resealable.
